(19) United States
(12) Patent Application Publication
(10) Pub. No.: US 2020/0165561 A1
(43) Pub. Date: May 28, 2020

(54) METHOD FOR PRODUCING PACELIOMYCES SATURATES MYCELIA, BIOPLASTIC AND METHOD FOR TREATING WASTEWATER

(71) Applicant: NATIONAL CHENG KUNG UNIVERSITY, Tainan City (TW)

(72) Inventors: Ten-chin Wen, Tainan City (TW); Chen-hsueh Lin, Tainan City (TW); Jiunn-jyi Lay, Tainan City (TW)

(21) Appl. No.: 16/202,137

(22) Filed: Nov. 28, 2018

(51) Int. Cl. C12N 1/14; C08B 37/08; C02F 3/34; C02F 1/66

(52) U.S. Cl. CPC C12N 1/14; C02F 1/66; C02F 3/347; C08B 37/003

(57) ABSTRACT

A method for producing mycelia of Paecilomyces saturatus, a bioplastic, and a method for treating wastewater are disclosed. The method for producing mycelia of Paecilomyces saturatus includes steps of: providing a culturing medium containing at least one monosaccharide and at least one inorganic substance; placing spores of Paecilomyces saturatus into the culturing medium; cultivating the spores of Paecilomyces saturatus in an aerobic environment for a predetermined period of time to grow mycelia of Paecilomyces saturatus; and filtering to obtain the mycelia of Paecilomyces saturatus.

### FIELD OF INVENTION

The present invention relates to a method for producing mycelia of Paecilomyces saturatus, a bioplastic, and a method for treating wastewater, and more particularly to a method for producing mycelia of Paecilomyces saturatus by various carbon sources and inorganic nitrogen sources to cultivate the mycelia of Paecilomyces saturatus, to a bioplastic formed by the mycelia of Paecilomyces saturatus, and to a method for treating wastewater using the mycelia of Paecilomyces saturatus.

### BACKGROUND OF INVENTION

With the development of industrial diversity, the traces of ammonia wastewater appear in a wide variety of industry such as the petrochemical, semiconductor, optoelectronics or wafer manufacturing industries. With the rising awareness of environmental protection among the general public, the limitation of ammonia nitrogen emission outside the water quality protection zone has been reduced to 30 ppm from the standard of 75 ppm in 2014, and the limit of ammonia nitrogen emission in the water quality protection zone must be or lower than 10 ppm.

At present, methods for treating ammonia-nitrogen wastewater can be divided into physical, chemical and biological methods by principles. Anammox anaerobic amine oxidation is the most widely used biological method, by which ammonia-nitrogen and nitrite-nitrogen can be simultaneously transformed to nitrogen gas in a bioreactor so that the ammonia-nitrogen can be removed. Compared with conventional biological method, the Anammox anaerobic amine oxidation saves many steps, but the disadvantage is that it is quite sensitive to the dissolved oxygen, pH value, temperature and the original microbial colonies in the wastewater. Furthermore, there will be much biological sludge generated after the treatment process, and the biological sludge still require a secondary treatment costs.

It is therefore necessary to provide a method for producing mycelia of Paecilomyces saturatus, a bioplastic, and a method for treating wastewater, in order to solve the problems existing in the conventional technology as described above.

### SUMMARY OF INVENTION

An object of the present invention is to provide a method for producing mycelia of Paecilomyces saturatus with various monosaccharides served as carbon sources and the maximum yield of the mycelia of Paecilomyces saturatus can be obtained by adjusting the ratio of the monosaccharides, so as to produce a large number of mycelia of Paecilomyces saturatus.

Another object of the present invention is to provide various applications of spores and mycelia of Paecilomyces saturatus, including a bioplastic and a method for treating wastewater. The bioplastic contains the mycelia of Paecilomyces saturatus as a major component and has biodegradable properties so that it is a green material. The method for treating wastewater is practiced by using the spores of Paecilomyces saturatus to degrade ammonia-nitrogen in the wastewater to grow the mycelia of Paecilomyces saturatus. The mycelia of Paecilomyces saturatus can adsorb the ammonia-nitrogen in the wastewater to concentrate the ammonia-nitrogen to a periphery of the spores of Paecilomyces saturatus, and therefore facilitate continuous degradation. Accordingly, in the presence of the ammonia-nitrogen, the spores of Paecilomyces saturatus can grow more mycelia, and the mycelia can assist the degradation of the ammonia-nitrogen. The method for treating wastewater is an environmental friendly, recyclable, and economical method.

To achieve above objects, one embodiment of the present invention provides a method for producing mycelia of Paecilomyces saturatus, comprising steps of: providing a culturing medium containing at least one monosaccharide and at least one inorganic substance; placing spores of Paecilomyces saturatus into the culturing medium; cultivating the spores of Paecilomyces saturatus in an aerobic environment for a predetermined period of time to grow mycelia of Paecilomyces saturatus; and filtering to obtain the mycelia of Paecilomyces saturatus.

In one embodiment of the present invention, the at least one monosaccharide is selected from a group consisting of glucose, fructose, and mannitol.

In one embodiment of the present invention, the at least one monosaccharide includes glucose, fructose, and mannitol; and the weight ratio of glucose, fructose, and mannitol is 1:3:1.

In one embodiment of the present invention, the predetermined period of time is equal to or more than 4 days.

In one embodiment of the present invention, the inorganic substance is selected from a group consisting of dipotassium phosphate, magnesium sulphate, sodium chloride, calcium sulphate, ferrous chloride, sodium molydbate, and ammonium chloride.

In one embodiment of the present invention, the culturing medium contains ammonium with a concentration equal to or more than 400 mg/L.

To achieve above objects, another embodiment of the present invention provides a bioplastic, comprising mycelia of Paecilomyces saturatus and a crosslinker, wherein the mycelia of Paecilomyces saturatus is prepared by the abovementioned method, and the mycelia of Paecilomyces saturatus is 90-98% of the bioplastic by weight.

In one embodiment of the present invention, the crosslinker is agar or genipin.

To achieve above objects, a further embodiment of the present invention provides a method for treating wastewater, comprising steps of: adding spores of Paecilomyces saturatus into an ammonia-nitrogen wastewater; and adjusting a pH value of the ammonia-nitrogen wastewater to be ranged from 4.8 to 5.2.

In one embodiment of the present invention, a suspension of the spores of Paecilomyces saturatus is firstly prepared, and then the suspension of the spores of Paecilomyces saturatus is added into the ammonia-nitrogen wastewater; wherein the suspension of the spores of Paecilomyces saturatus has a concentration of 10⁶ CFU/ml in the suspension, and the suspension of the spores of Paecilomyces saturatus is equal to or more than 1% of the ammonia-nitrogen wastewater by weight.

### BRIEF DESCRIPTION OF DRAWINGS

FIGS. 1A-1C show the classical phenotype characteristic of Paecilomyces saturatus strains in the present invention observed by a microscope.

FIG. 2 shows dried biomass distribution of three different monosaccharide ratios in Experiment 3 of the present invention.

FIG. 3 shows the trend of a concentration of ammonium in Experiment 4 of the present invention under different cultivating time.

FIG. 4 shows the mycelia of Paecilomyces saturatus after stimulated wastewater treatment in Experiment 4.

FIG. 5 shows the appearance of the bioplastic film formed by agar and mycelia with different ratios.

FIG. 6 shows the appearance of the bioplastic film produced in Experiment 8.

FIG. 7 shows that the azo dyes in Experiment 9 of the present invention are adsorbed on the fungal cell wall of the mycelia.

FIG. 8 shows the changes of the UV-Vis spectrum and the color before and after degradation of Sudan Black B in Experiment 10 of the present invention.

### DETAILED DESCRIPTION OF PREFERRED EMBODIMENTS

The above and other objects, features, and advantages can be best understood by referring to the following detailed description of the preferred embodiments and the accompanying drawings. Furthermore, if there is no specific description in the invention, singular terms such as “a”, “one”, and “the” include the plural number. If there is no specific description in the invention, the numerical range (e.g. 10%-11% of A) contains the upper and lower limit (i.e. 10%≤A≤11%). If the lower limit is not defined in the range (e.g. less than, or below 0.2% of B), it means that the lower limit may be 0 (i.e. 0%≤B≤0.2%). The abovementioned terms are used to describe and understand the present invention, but the present invention is not limited thereto.

One embodiment of the present invention provides a method for producing mycelia of Paecilomyces saturatus, comprising steps of: providing a culturing medium containing at least one monosaccharide and at least one inorganic substance; placing spores of Paecilomyces saturatus into the culturing medium; cultivating the spores of Paecilomyces saturatus in an aerobic environment for a predetermined period of time to grow mycelia of Paecilomyces saturatus; and filtering to obtain the mycelia of Paecilomyces saturatus. In one embodiment, the at least one monosaccharide is selected from a group consisting of glucose, fructose, and mannitol. Preferably, the at least one monosaccharide includes glucose, fructose, and mannitol; and the weight ratio of glucose, fructose, and mannitol is 1:3:1. In one embodiment of the present invention, the predetermined period of time is equal to or more than 4 days. In one embodiment of the present invention, the inorganic substance is selected from a group consisting of dipotassium phosphate, magnesium sulphate, sodium chloride, calcium sulphate, ferrous chloride, sodium molydbate, and ammonium chloride. In one embodiment of the present invention, the culturing medium contains ammonium with a concentration equal to or more than 400 mg/L.

Another embodiment of the present invention provides a bioplastic, comprising mycelia of Paecilomyces saturatus and a crosslinker, wherein the mycelia of Paecilomyces saturatus is prepared by the abovementioned method, and the mycelia of Paecilomyces saturatus is 90-98% of the bioplastic by weight. In one embodiment of the present invention, the crosslinker is agar or genipin.

A further embodiment of the present invention provides a method for treating wastewater, comprising steps of: adding spores of Paecilomyces saturatus into an ammonia-nitrogen wastewater; and adjusting a pH value of the ammonia-nitrogen wastewater to be ranged from 4.8 to 5.2. In one embodiment, a suspension of the spores of Paecilomyces saturatus is firstly prepared, and then the suspension of the spores of Paecilomyces saturatus is added into the ammonia-nitrogen wastewater, wherein the suspension of the spores of Paecilomyces saturatus has a concentration of 10⁶ CFU/ml in the suspension, and the suspension of the spores of Paecilomyces saturatus is equal to or more than 1% of the ammonia-nitrogen wastewater by weight.

To verify the composition of the mycelia of Paecilomyces saturatus and the effects in the applications of the mycelia of Paecilomyces saturatus, the following experiments were conducted.

### Experiment 1: Cultivation of Strains of Paecilomyces saturatus

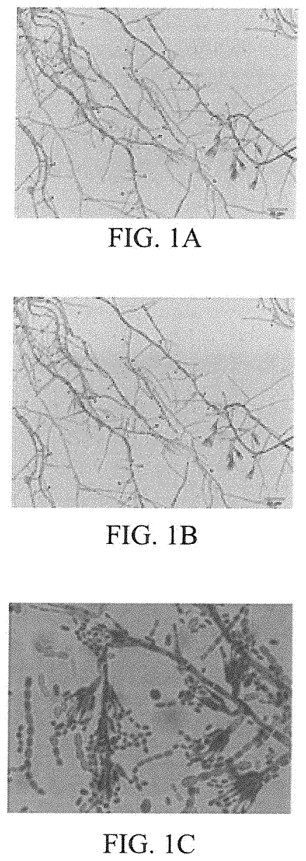

First, 40 ppm of a symbiotic fermentation broth was inoculated in a culture medium containing high concentration of ammonium. After culturing for 14 days under aerobic aeration, fungal strains were screened from the environment. Then, the fungal strains were cultivated at 37° C. for 7 days. Next, the green-brown colonies I on the agar plate were observed with naked eye. Alternatively, the fungal strains were screened from wastewater of industrial sewage treatment plant in which the wastewater contains ammonium ions and then the fungal strains were cultivated at 37° C. for 7 days. Next, the green-brown colonies II on the agar plate were observed with naked eye. Mycelia of the green-brown colonies I and the green-brown colonies II were respectively immobilized from the agar plate to the slide glass and stained with Coomassie Brilliant Blue. They both show broom-like conidiophores producing spherical asexual spores under the microscope that is a classical phenotype characteristic of Paecilomyces genius as shown in FIGS. 1A to 1C, which show the classical phenotype characteristic of the green-brown colonies II observed by a microscope.

### Experiment 2: Identification of Paecilomyces saturatus

The green-brown colonies I and green-brown colonies II obtained in Experiment 1 were identified by ITS sequencing, and then the fungal strains cultured in Experiment 1 were confirmed to have 99.9% similarity with the fungal strain Paecilomyces saturatus. Thus, it can be confirmed that the strains obtained in Experiment 1 is Paecilomyces saturatus which has been deposited in the ATCC by professor Nakazawa (ATCC 11971™). In addition, both strains in Experiment 1 have same characteristics as ATCC 11971™, they can grow in the liquid medium and use acetic acid as a single carbon source. Although the sources of the fungal strains obtained in Experiment 1 were different, both of the fungal strains are Paecilomyces saturatus.

### Experiment 3: Cultivation of Mycelia

A medium containing 5 g/L carbon source (monosaccharide: glucose, fructose, and/or mannitol), 1 g/L dipotassium phosphate, 0.5 g/L magnesium sulphate, 0.5 g/L sodium chloride, 1 g/L calcium sulphate, 40 mg/L ferrous chloride, 5 mg/L sodium molydbate, and 0.764 g/L ammonium chloride is prepared. After 1 L medium was sterilized under 121° C. for an hour, 1 wt % of a suspension of spores of Paecilomyces saturatus was added into a serum bottle.

After aerobic culturing for 4 days, the mycelia was collected by filtration and overall dried biomass is listed in the table 1.

TABLE 1

| | glucose | fructose | mannitol | dried biomoass |
|---|---|---|---|---|
| Group | (g/L) | (g/L) | (g/L) | (g/L) |
| 1 | 5 | 0 | 0 | 0.643 |
| 2 | 0 | 5 | 0 | 2.638 |
| 3 | 0 | 0 | 5 | 2.817 |
| 4 | 1.66 | 1.66 | 1.66 | 0.398 |
| 5 | 2.5 | 2.5 | 0 | 2.598 |
| 6 | 0 | 2.5 | 2.5 | 2.792 |
| 7 | 2.5 | 0 | 2.5 | 2.695 |
| 8 | 3.32 | 1.66 | 1.66 | 1.23 |
| 9 | 1.66 | 3.32 | 1.66 | 3.266 |
| 10 | 1.66 | 1.66 | 3.32 | 3.424 |
| 11 | 3.75 | 1.25 | 0 | 0.814 |
| 12 | 1.25 | 3.75 | 0 | 1.919 |
| 13 | 3.75 | 0 | 1.25 | 3.251 |
| 14 | 1.25 | 0 | 3.75 | 3.219 |
| 15 | 0 | 3.75 | 1.25 | 2.34 |
| 16 | 0 | 1.25 | 3.75 | 2.271 |
| 17 | 1 | 3 | 1 | 3.501 |

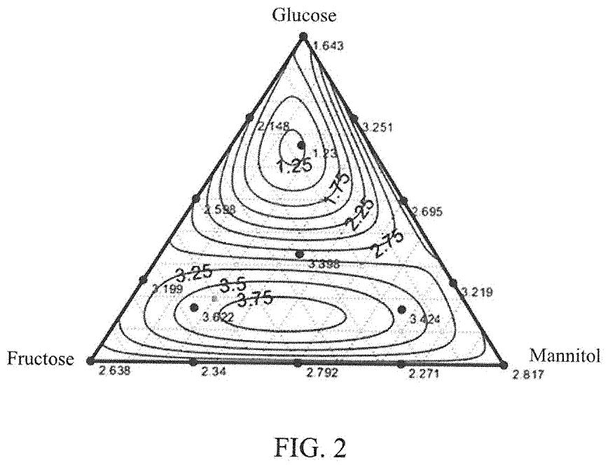

From Table 1, the maximum yield of the dried biomass can be obtained by the monosaccharide used in group 17. The optimal ratio of the carbon source is (glucose:fructose:mannitol=1:3:1). Under this ratio, 3.501 g/L of the dried biomass can be obtained as shown in FIG. 2.

### Experiment 4: Simulation of Treating Ammonia-Nitrogen Wastewater

In 1 L batch aerobic bioreactor, initial ammonium concentration is controlled to be 400 mg/L, temperature is controlled to be 25° C., pH is controlled to be 5. The aerobic bioreactor is agitated by an air distributor from the bottom of the reactor. After 1% of the suspension of spores of Paecilomyces saturatus (10⁶ CFU/ml) by weight was added into the reactor and cultured for 4 days, and then standing for 12 hours. The ammonium concentration in water was continuously monitored.

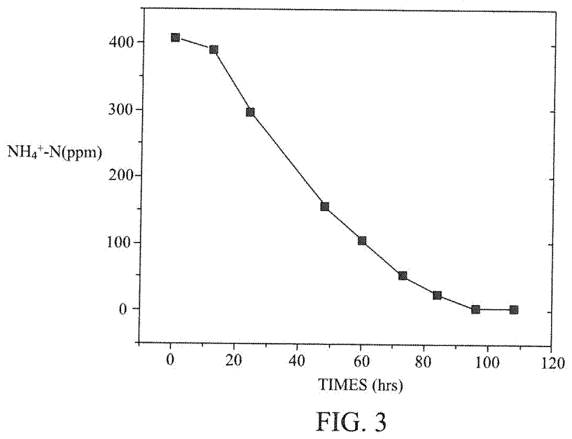

Refer to FIG. 3, the ammonium concentration was decreased from 406.2 ppm to 1.82 ppm throughout the experiment. The removal rate of ammonium obtained by calculating is 99.5%, so that the drainage requirement of wastewater can be reached.

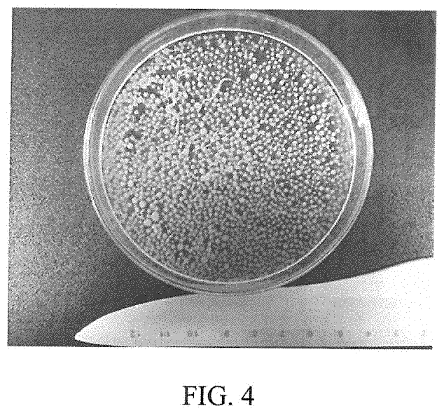

Additionally, the final mycelia biomass is a pellet like form which can be easily settling after the bio-treatment process as e shown in FIG. 4.

### Experiment 5: Extraction of Chitin/Chitosan

After spores of Paecilomyces saturatus were cultured in a liquid medium under aerobic condition for 4 days, the mycelia was harvested by gravity filtration and dried in vacuum oven overnight. Raw biomass was finely grinded and washed with 0.5M NaOH solution under 120° C. for 0.5 hours. Centrifuging the alkali solution at 8000 rpm for 10 mins and the supernatant was removed to obtain a precipitation A. The precipitation A was taken out and re-dissolved in 10 wt % acetic acid under 120° C. for 4 hours. Centrifuging the acid solution at 8000 rpm for 10 mins and respectively collect a supernatant B and a precipitation C. The pH of the supernatant B is manipulated to 8 with dropwise addition of 150 mM NaOH. Finally, a precipitation D on the bottom was collected by centrifugation. After freeze drying the precipitation D overnight, a pure fungal chitosan can be obtained. After freeze drying the precipitation C for 8 hours, a pure fungal chitin can be obtained.

The DD % (degree of deacetylation) of chitosan extracted from mycelia of Paecilomyces saturatus is about 84%, which shows that Paecilomyces saturatus is indeed a promising source for high DD % chitosan. The crystallinity index of the chitosan calculating from the XRD spectrum is about 3.2.

Chitin/Chitosan is widely existing in the shell of crustacean creatures. It is usually necessary to extract the chitosan using strong mechanical force, concentrated alkali solution, and extremely high temperature. Eventually the physical properties of the crustacean chitosan turn out to be low molecular weight and low deacetylation ratio, which are not good enough. In addition, the chitin derived from the extraction is also low molecule weight. In contrast, the present invention uses Paecilomyces saturatus as a source for extracting chitosan and chitin. Since cell wall of the Paecilomyces saturatus is rich in chitin/chitosan, and the network-liked fungal mycelia is loosely composed of microfibrillar, the chitosan can be easily extracted from the cell wall by a gentle method, as well as high molecular weight and high deacetylation rate can be maintained. Therefore, Paecilomyces saturatus is an excellent source of high quality chitin/chitosan.

### Experiment 6: Production of Bioplastic Film by Mycelia

0.2 g dried mycelia of Paecilomyces saturatus was well rinsed in 10 ml deionized water and heated toward 100° C. holding for 15 mins. After the heating process was done, the homogeneous solution was poured into a mold and cooled to room temperature. Then the mold is dried in an oven for 8 hours under 37° C. A mycelia film can be obtained.

### Experiment 7: Production of Bioplastic Film by Mycelia and Agar

0.1 g dried mycelia of Paecilomyces saturatus and different ratio of agar were well rinsed in 10 ml deionized water and heated toward 100° C. holding for 15 mins. After the heating process was done, the homogeneous solution was poured into a mold and cooled to room temperature. Then the mold was dried in an oven for 8 hours under 37° C. A mycelia/agar film can be obtained with 1 to 3% water content. It is also possible to replace agar with gelatin or hyaluronic acid to form hydrogen bonds with hydroxyl group in the molecular chain for a physical crosslinking reaction.

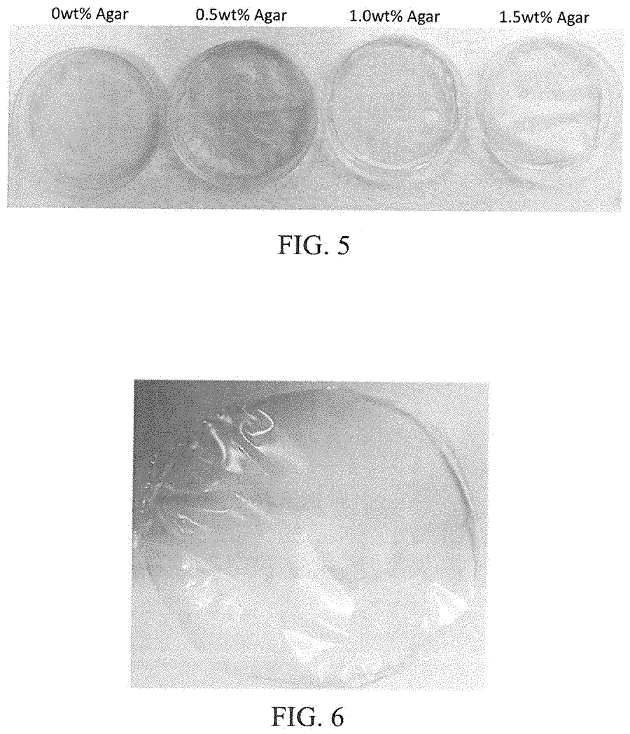

As shown in FIG. 5, the mechanical strength of the mycelia film is weak and fragile without the addition of agar (0 wt %). However, the strength has been significantly improved with 0.5 wt % addition of agar. As the gradually increase of the agar by the ratio from 0.5 wt % to 1.5 wt %, the obtained bioplastic film become smoother and tougher, and the hydrophobicity of the bioplastic film is also increased.

### Experiment 8: Production of Bioplastic Film by Mycelia and Genipin

After 0.2 g dried mycelia of Paecilomyces saturatus was well rinsed in 10 ml deionized water, 4 mg of genipin was dissolved in precursor solution and a crosslinking reaction was carried out for 4 hours. After the crosslinking reaction was done, the homogeneous solution was poured into a mold and cooled to room temperature. Then the mold was dried in an oven for 8 hours under 37° C. to obtain a mycelia/genipin film as shown in FIG. 6 with 1 to 3% water content. It is possible to replace genipin with glutaraldehyde or maleic anhydride to crosslink with amine group in the molecular chain of the mycelia by a dehydration polymerization.

### Experiment 9: Adsorption of Dyes by Mycelia

An azo dye of Sudan Black B (SBB) and water were mixed to form 150 mg/L SBB aqueous solution. Then 2 wt % dried mycelia of Paecilomyces saturatus was added and the pH value was adjusted with dropwise NaOH. To observe the effect of pH on adsorption, the pH value was changed from 3 to 9 and the saturated adsorption effect of the mycelia of Paecilomyces saturatus on the dye was recorded.

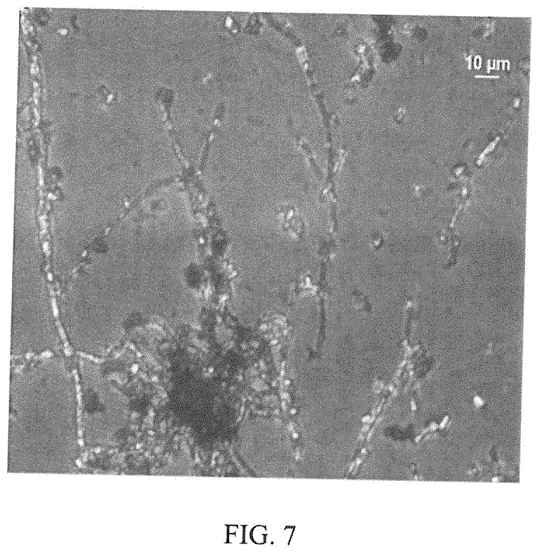

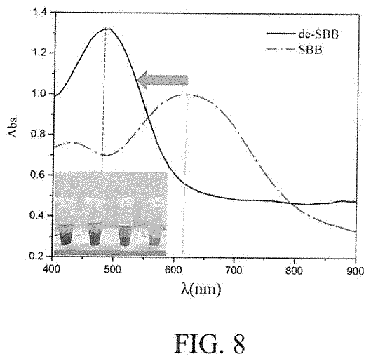

When pH is 7, the ammonium concentration of the SBB aqueous solution can rapidly drop to 19.8 mg/L at the first 3 hours, and the overall azo dye removal was 94%. Therefore, the mycelia of Paecilomyces saturatus can be an ideal bio-adsorbent material for azo dye treatment. In FIG. 7, it is clear that the azo dye adsorbs on the fungal cell wall of the mycelia.

Azo dyes are organic compounds containing C—N═N—C linkage and aromatic rings. They are widely used in textile and leather articles industry. Since the cell wall of the mycelia of Paecilomyces saturatus is rich of hydroxyl group, amine group, sulfate group, and phosphate group which can enhance the adsorption capability to the azo dyes, and the mycelia can be served as a good bio-adsorbent.

### Experiment 10: Degradation of Dyes by Mycelia

0.2 wt % dried mycelia of Paecilomyces saturatus was weighted and swelled in 100 ml sterilized DI water. Then 1 wt % Sudan Black B (SBB) ethanol solution was prepared and stirred for 30 mins until it dissolved uniformly. Finally, 2 ml SBB ethanol solution was added into 98 ml mycelia solution in erlenmeyer flak and pH of the solution was adjusted to 7 with NaOH and acetic acid. The flask had been shaken under 80 rpm at 37° C. for two weeks.

SBB will be adsorbed on the mycelia initially and turn to dark blue. As time goes on, the dark blue mycelia will turn to purple or even pink. From the UV-vis spectrum, the SBB adsorption peak around 600 nm was disappeared and popped out a new adsorption zone around 500 nm after degradation, and this result indicates that the molecular structure of SBB has been changed as shown in FIG. 8. From the blue shift of the characteristic absorption peak, the SBB must be degraded toward smaller molecules which provide shorter conjugate electron pathway system.

Compared with the prior art, in the method for treating wastewater provided by the present invention, the Paecilomyces saturatus is a kind of fungus, and the ammonia-nitrogen wastewater is treated in an absolutely aerobic environment, so that the dissolved oxygen in the water is not strict. The fungus biomass derived from the treatment can be easily separated from the mixture, and the economical fungal mycelia by-product can be directly recovered for further application to preparation of a bioplastic or a bio-adsorbent.

The present disclosure has been described with preferred embodiments thereof and it is understood that many changes and modifications to the described embodiments can be made by a skilled person in the art without departing from the scope and the spirit of the disclosure that is intended to be limited only by the appended claims.

1. A method for treating wastewater, comprising steps of:
adding spores of Paecilomyces saturatus into an ammonia-nitrogen wastewater; and
adjusting a pH value of the ammonia-nitrogen wastewater to be ranged from 4.8 to 5.2;
wherein the ammonia-nitrogen wastewater has an initial ammonium concentration controlled to be greater or equal to 400 mg/L.

2. The method according to claim 1, wherein a suspension of the spores of Paecilomyces saturatus is firstly prepared, and then the suspension of the spores of Paecilomyces saturatus is added into the ammonia-nitrogen wastewater, wherein the suspension of the spores of Paecilomyces saturatus has a concentration of 10⁶ CFU/ml in the suspension, and the suspension of the spores of Paecilomyces saturatus is equal to or more than 1% of the ammonia-nitrogen wastewater by weight.
